(19) United States
(12) Patent Application Publication
(10) Pub. No.: US 2025/0082308 A1
(43) Pub. Date: Mar. 13, 2025

(54) ULTRASOUND DIAGNOSTIC SYSTEM AND ULTRASOUND DIAGNOSTIC APPARATUS

(71) Applicant: FUJIFILM Corporation, Tokyo (JP)

(72) Inventors: Suguru ISHIGURO, Tokyo (JP); Eiji KASAHARA, Tokyo (JP); Akira KUSAKABE, Tokyo (JP); Yu ZHAO, Tokyo (JP)

(73) Assignee: FUJIFILM Corporation, Tokyo (JP)

(21) Appl. No.: 18/827,833

(22) Filed: Sep. 8, 2024

(30) Foreign Application Priority Data
Sep. 11, 2023 (JP) 2023-146804

(51) Int. Cl. A61B 8/00; A61B 8/08

(52) U.S. Cl. CPC A61B 8/56; A61B 8/464; A61B 8/48; A61B 8/54

(57) ABSTRACT

A management table has management information representing interrelationships between a plurality of APs constituting an AP group. An AP management unit manages selection of an AP set from the AP group and manages an operation of the AP set, based on the management information. The AP set consists of a plurality of APs that operate together during an ultrasound examination. The management information includes a plurality of pieces of proper combination information. Each piece of the proper combination information includes a designation of a parallel operation or a designation of a serial operation.

### CROSS-REFERENCE TO RELATED APPLICATION

This application claims the priority benefit of Japan application serial no. 2023-146804, filed on Sep. 11, 2023. The entirety of the above-mentioned patent application is hereby incorporated by reference herein and made a part of this specification.

### BACKGROUND OF THE INVENTION

### 1. Field of the Invention

The present disclosure relates to an ultrasound diagnostic system and an ultrasound diagnostic apparatus, and particularly, to management of operations of a plurality of application programs (APs).

### 2. Description of the Related Art

An ultrasound diagnostic apparatus is used in an ultrasound examination. With enhancement of functions of the ultrasound diagnostic apparatus, there is an increasing information processing load on the ultrasound diagnostic apparatus. Against such a background, an ultrasound diagnostic system consisting of an ultrasound diagnostic apparatus and an information processing apparatus (hereinafter, referred to as an external information processing apparatus) has been proposed (refer to, for example, JP2022-165214A and JP2022-167500A).

The external information processing apparatus includes an AP for processing image data generated by the ultrasound diagnostic apparatus. Specifically, the AP is software for applying specific processing to the image data. A processing result of the AP is displayed on the ultrasound diagnostic apparatus. Examples of the specific processing executed by the AP include analysis of the image data, processing of the image data, and measurement based on the image data. The specific processing may include other processing to be applied to a result of processing the image data.

In the specification of the present application, in some cases, the AP included in the external information processing apparatus is referred to as an external AP, and the AP included in the ultrasound diagnostic apparatus is referred to as an internal AP.

### SUMMARY OF THE INVENTION

In order to support the ultrasound examination, it is desired to provide more valuable information to an examiner. For that purpose, it is desired to simultaneously operate a plurality of APs during the ultrasound examination, or it is desired to cooperate the plurality of APs. However, the conventional ultrasound diagnostic system does not have a mechanism for operating the plurality of APs including the external AP together.

An object of the present disclosure is to operate a plurality of APs including an external AP together in an ultrasound diagnostic system. Alternatively, the object of the present disclosure is to properly operate the plurality of APs including the external AP in the ultrasound diagnostic system.

According to the present disclosure, there is provided an ultrasound diagnostic system comprising: an ultrasound diagnostic apparatus that generates image data based on reception data; an external information processing apparatus connected to the ultrasound diagnostic apparatus; an application program (AP) group provided in the external information processing apparatus or provided across the ultrasound diagnostic apparatus and the external information processing apparatus, and composed of a plurality of APs for processing the image data; and a management unit that manages selection of an AP set from the AP group and manages an operation of the AP set, based on management information representing interrelationships between the plurality of APs constituting the AP group, in which the AP set consists of a plurality of APs that operate together during an ultrasound examination.

According to the present disclosure, there is provided an ultrasound diagnostic apparatus connected to an external information processing apparatus, the ultrasound diagnostic apparatus comprising: a generation unit that generates image data based on reception data; and a management unit that manages an application program (AP) group composed of a plurality of APs for processing the image data, in which the AP group is provided in the external information processing apparatus or is provided across the ultrasound diagnostic apparatus and the external information processing apparatus, the management unit manages selection of an AP set from the AP group and manages an operation of the AP set, based on management information representing interrelationships between the plurality of APs constituting the AP group, and the AP set consists of a plurality of APs that operate together during an ultrasound examination.

According to the present disclosure, there is provided a program executed in an ultrasound diagnostic system, the ultrasound diagnostic system including an ultrasound diagnostic apparatus that generates image data based on reception data, and an external information processing apparatus connected to the ultrasound diagnostic apparatus, in which an application program (AP) group composed of a plurality of APs for processing the image data is provided in the external information processing apparatus or is provided across the ultrasound diagnostic apparatus and the external information processing apparatus, the program comprising: a function of managing selection of an AP set from the AP group based on management information representing interrelationships between the plurality of APs constituting the AP group; and a function of managing an operation of the AP set based on the management information, in which the AP set consists of a plurality of APs that operate together during an ultrasound examination.

According to the present disclosure, in the ultrasound diagnostic system, it is possible to operate the plurality of APs including the external AP together. Alternatively, according to the present disclosure, in the ultrasound diagnostic system, it is possible to properly operate the plurality of APs including the external AP.

### BRIEF DESCRIPTION OF THE DRAWINGS

FIG. 1 is a diagram showing a configuration example of an ultrasound diagnostic system according to an embodiment.

FIG. 2 is a diagram showing a configuration example of an AP management unit.

FIG. 3 is a diagram showing an example of an AP list.

FIG. 4 is a diagram showing a configuration example of management information.

FIG. 5 is a diagram showing a first example of a management table.

FIG. 6 is a diagram showing a second example of the management table.

FIG. 7 is a diagram showing an initial state of a selection image.

FIG. 8 is a diagram showing a first state of the selection image.

FIG. 9 is a diagram showing a second state of the selection image.

FIG. 10 is a diagram showing a third state of the selection image.

FIG. 11 is a diagram showing a fourth state of the selection image.

FIG. 12 is a diagram showing an example of a parallel operation of a plurality of APs.

FIG. 13 is a diagram showing a first example of a serial operation of a plurality of APs.

FIG. 14 is a diagram showing a second example of the serial operation of the plurality of APs.

FIG. 15 is a diagram showing a first example of a result image.

FIG. 16 is a diagram showing a first example of an operation image.

FIG. 17 is a diagram showing a second example of the result image.

FIG. 18 is a diagram showing a second example of the operation image.

FIG. 19 is a diagram showing another configuration example of the ultrasound diagnostic system according to the embodiment.

### DESCRIPTION OF THE PREFERRED EMBODIMENTS

Hereinafter, embodiments will be described with reference to the drawings.

### (1) Outline of Embodiment

An ultrasound diagnostic system according to an embodiment includes an ultrasound diagnostic apparatus, an external information processing apparatus, an AP group, and a management unit. The ultrasound diagnostic apparatus generates image data based on reception data. The external information processing apparatus is connected to the ultrasound diagnostic apparatus. The AP group is composed of a plurality of APs for processing the image data. The AP group is provided in the external information processing apparatus or is provided across the ultrasound diagnostic apparatus and the external information processing apparatus. The management unit manages selection of an AP set from the AP group and manages execution of the AP set, based on management information representing interrelationships between the plurality of APs constituting the AP group. The management unit can also be referred to as an AP manager. The AP set consists of a plurality of APs that operate together during an ultrasound examination.

With the above-described configuration, the AP set can be correctly selected, and the AP set can be correctly operated, based on the management information. Therefore, it is possible to provide more information or more valuable information to an examiner.

The AP set is composed of a plurality of APs that can operate simultaneously or a plurality of APs that can operate in cooperation. The plurality of APs may be simultaneously activated, or the plurality of APs may be activated in a stepwise manner. The AP set includes at least one external AP. Alternatively, the AP set is composed of a plurality of the external APs. In the embodiment, the AP set is selected by a user before the ultrasound examination. The selection can be referred to as package selection of the plurality of APs. The AP set may be automatically selected.

In the embodiment, the management information includes one or a plurality of pieces of proper combination information, and each piece of the proper combination information includes information for specifying the plurality of APs that operate together. The management information is generated by the user or is automatically generated based on collected information.

In the embodiment, each piece of the proper combination information further includes a designation of a parallel operation or a designation of a serial operation. The management unit causes the plurality of APs constituting the AP set to perform the parallel operation in accordance with the designation of the parallel operation and causes the plurality of APs constituting the AP set to perform the serial operation in accordance with the designation of the serial operation. With this configuration, in accordance with the interrelationships between the plurality of APs constituting the AP set, it is possible to properly operate the APs. The parallel operation means that the plurality of APs do not have a data exchange relationship and simultaneously operate in parallel without cooperating with each other. The serial operation means that the plurality of APs have a data exchange relationship and operate simultaneously or non-simultaneously operate in cooperation with each other.

In the embodiment, the proper combination information including the designation of the serial operation includes information indicating a connection order of the plurality of APs constituting the AP set. The management unit connects the plurality of APs constituting the AP set in series in accordance with the information indicating the connection order. With this configuration, it is possible to correctly connect the plurality of APs.

In the embodiment, the management information has a matrix structure. The matrix structure includes a plurality of cells corresponding to a plurality of AP pairs, which are a plurality of combinations defined based on the AP group. The plurality of cells consist of one or a plurality of valid cells associated with one or a plurality of pieces of proper combination information, and one or a plurality of invalid cells other than the one or the plurality of valid cells. With this configuration, various interrelationships in the AP group are easily managed. The AP set consists of a single AP pair or consists of three or more APs specified by a plurality of AP pairs, or corresponds to a sequence of a plurality of AP pairs.

In the embodiment, the matrix structure includes a first axis to which a plurality of AP candidates to be selected earlier are assigned and a second axis to which a plurality of AP candidates to be selected later are assigned. The management unit identifies a specific cell based on an AP selected earlier and an AP selected later in the matrix structure and refers to information associated with the specific cell.

In the embodiment, the management unit connects, in controlling the serial operation of the plurality of APs constituting the AP set, the plurality of APs constituting the AP set in series in an order according to the management information so that processing result data output from an i-th AP is input to an i+1-th AP.

The processing result data is composed of image data, numerical data, and the like. In a case in which the number of APs constituting the AP set is represented by n (where n is an integer of 2 or more), i is an integer of 1 or more and n−1 or less. For example, in a case in which n is 3, i takes 1 and 2.

In the embodiment, the ultrasound diagnostic apparatus includes the management unit. The management unit receives the processing result data output from the i-th AP and transfers the processing result data to the i+1-th AP.

In the embodiment, the external information processing apparatus includes a buffer memory. The management unit manages an operation of the external information processing apparatus such that, in a case in which the i-th AP and the i+1-th AP belong to the external information processing apparatus, the processing result data output from the i-th AP is written into the buffer memory and the processing result data read from the buffer memory is input to the i+1-th AP. With this configuration, an amount of data transferred between the ultrasound diagnostic apparatus and the external information processing apparatus can be reduced.

The ultrasound diagnostic system according to the embodiment includes a selection image generation unit that generates a selection image for selecting the AP set from the AP group, based on the management information. In the embodiment, the selection image includes one or a plurality of display elements indicating one or a plurality of AP candidates that are currently selectable. A content of the selection image changes in a stepwise manner in a process of selecting a plurality of APs from the AP group in a stepwise manner. The AP set can be easily selected by narrowing down the selectable APs.

The ultrasound diagnostic system according to the embodiment includes a display controller that controls display of a plurality of pieces of processing result data output from the plurality of APs constituting the AP set. The display controller displays the plurality of pieces of processing result data in a juxtaposed manner in a case in which a juxtaposed display mode is selected, and displays the plurality of pieces of processing result data in a composite manner in a case in which a composite display mode is selected. With this configuration, it is possible to display a plurality of processing results in a display format desired by the user.

### (2) Details of Embodiment

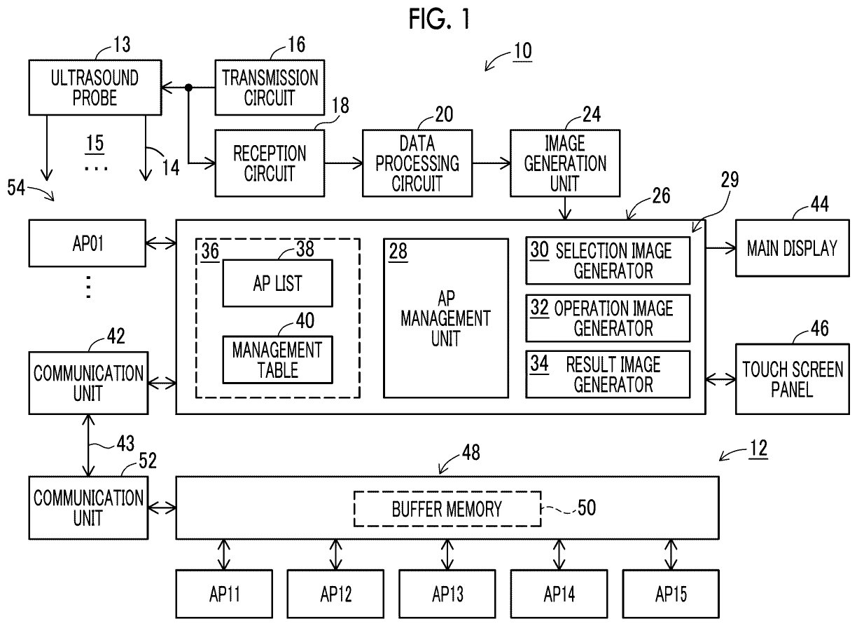

FIG. 1 shows an ultrasound diagnostic system according to the embodiment. The ultrasound diagnostic system is a medical system installed in a medical institution such as a hospital and used in an ultrasound examination of a living body. The ultrasound diagnostic system is composed of an ultrasound diagnostic apparatus 10 and an external information processing apparatus 12. The external information processing apparatus 12 is connected to the ultrasound diagnostic apparatus 10 via a dedicated cable 43. The external information processing apparatus 12 may be connected to the ultrasound diagnostic apparatus 10 via a network such as a local area network (LAN) or the Internet.

First, a configuration of the ultrasound diagnostic apparatus 10 will be described. An ultrasound probe 13 is a device that transmits ultrasound waves into the living body and that receives reflected waves from the living body. The ultrasound probe 13 includes a transducer array consisting of a plurality of transducers. The transducer array forms an ultrasound beam 14. A beam scanning plane 15 is formed through electronic scanning of the ultrasound beam 14.

As an electronic scanning method of the ultrasound beam 14, an electronic linear scanning method, an electronic sector scanning method, and the like are known. A two-dimensional transducer array may be provided as the transducer array. Volume data can be acquired from a three-dimensional space in the living body by performing two-dimensional scanning with the ultrasound beam through the two-dimensional transducer array.

A transmission circuit 16 is an electronic circuit functioning as a transmission beam former and outputs a plurality of transmission signals to the transducer array in parallel during transmission. Consequently, a transmission beam is formed.

A reception circuit 18 is an electronic circuit functioning as a reception beam former and applies phase addition to a plurality of reception signals output in parallel from the transducer array during reception, thereby generating reception beam data. With repetition of the electronic scanning of the ultrasound beam, a reception frame data sequence is output from the reception circuit 18. Each reception frame data is composed of a plurality of pieces of reception beam data that are arranged in an electronic scanning direction. Each reception beam data is composed of a plurality of pieces of echo data that are arranged in a depth direction.

A data processing circuit 20 is a circuit that processes each reception frame data. The data processing circuit 20 includes a detection circuit, a logarithmic transformation circuit, a filter, and the like. In addition, the data processing circuit 20 also includes a circuit that extracts and processes Doppler information included in each reception beam data.

An image generation unit 24 includes a digital scan converter (DSC). The DSC has a coordinate transformation function, a pixel interpolation function, and the like. A display frame data sequence is generated from the reception frame data sequence by the DSC. More specifically, for example, in a case in which a B-mode is selected, the image generation unit 24 generates a tomographic image data sequence as the display frame data sequence. In a case in which a color flow mapping (CFM) mode is selected, the image generation unit 24 generates the tomographic image data sequence and a blood flow image data sequence as the display frame data sequence. An operation mode other than the B-mode and the CFM mode may be selected.

A cine memory (not shown) is provided following the image generation unit 24. The cine memory has a ring buffer structure. The cine memory temporarily stores the tomographic image data sequence and/or the blood flow image data sequence. The image data sequence (the tomographic image data sequence and/or the blood flow image data sequence) read from the cine memory is sent to an information processing unit 26.

The image data sequence is sent from the information processing unit 26 to a main display 44. The ultrasound image is displayed on the main display 44. In a case in which the B-mode is selected, a tomographic image is displayed on the main display 44, and in a case in which the CFM mode is selected, a CFM image is displayed on the main display 44. The CFM image is generated by combining the tomographic image data sequence and the blood flow image data sequence. In a real-time operation state, the tomographic image and the CFM image are each a moving image. In a frozen state (in a state in which the transmission and reception are temporarily stopped), the tomographic image and the CFM image are each a still image. Another ultrasound image may be displayed on the main display 44.

The information processing unit 26 is configured with a processor that executes a program. The processor is specifically a central processing unit (CPU). The information processing unit 26 according to the embodiment functions as a controller that controls operations of a plurality of elements constituting the ultrasound diagnostic apparatus 10. In FIG. 1, a plurality of representative functions exhibited by the information processing unit 26 are represented by a plurality of blocks. Specifically, the information processing unit 26 functions as an AP management unit 28, a selection image generator 30, an operation image generator 32, and a result image generator 34.

The ultrasound diagnostic system according to the embodiment includes an application program (AP) group 54 consisting of a plurality of APs for processing the image data generated by the ultrasound diagnostic apparatus 10. In the embodiment, the AP group 54 consists of a plurality of external APs (an AP11 to an AP15 to be described below) and one internal AP (AP01). A plurality of the internal APs may be provided in the ultrasound diagnostic apparatus 10.

Hereinafter, a configuration and an operation of the ultrasound diagnostic system will be described assuming that a plurality of APs including at least one external AP are operated together during the ultrasound examination as a premise. However, in the ultrasound diagnostic system, only a single AP may be activated during the ultrasound examination.

The AP management unit 28 is a module that manages the AP group 54, and is a module that specifically manages the selection of the AP set from the AP group 54 and the operation of the AP set. Here, the AP set consists of a plurality of APs that operate together in the ultrasound examination as described above. In the embodiment, the AP set includes at least one external AP. The AP set can also be referred to as a software set. The AP management unit 28 corresponds to a main interface module for the AP group 54.

The AP management unit 28 also has a function of collecting information necessary for managing the AP group, a function of managing the operation of the external information processing apparatus, a function of managing data transfer, and the like. The selection image generator 30, the operation image generator 32, and the result image generator 34 correspond to a display processing unit 29. The information processing unit 26 may function as an examination report creation unit.

In FIG. 1, a memory 36 connected to the information processing unit 26 is represented as a part of the information processing unit 26 for convenience. The memory 36 stores an AP list 38 and a management table 40. The AP list 38 is for managing the AP group, and the management table 40 has management information representing interrelationships between the plurality of APs constituting the AP group.

The main display 44 and a touch screen panel 46 are connected to the information processing unit 26. The main display 44 is configured with a liquid crystal display, an organic EL display device, or the like. The touch screen panel 46 is provided on an operation panel (not shown). The touch screen panel 46 is composed of a screen panel and a touch panel. In the embodiment, the ultrasound image and the result image are displayed on the main display 44, and the operation image and the selection image are displayed on the touch screen panel 46. A communication unit 42 is connected to the information processing unit 26. The information processing unit 26 may function as the communication unit 42.

In the configuration example shown in FIG. 1, the AP01 is connected to the information processing unit 26. The AP01 is the internal AP. Although the AP01 is software executed by the processor, in FIG. 1, the AP01 is represented outside the information processing unit 26.

Next, a configuration of the external information processing apparatus 12 will be described. The external information processing apparatus 12 is a general-purpose information processing apparatus or a dedicated information processing apparatus.

The external information processing apparatus 12 includes an information processing unit 48. The information processing unit 48 is configured with a processor that executes a program, and the processor is, for example, a CPU. A plurality of APs are connected to the information processing unit 48, specifically, the AP11, the AP12, the AP13, the AP14, the AP15 are connected. The APs are each executed by the processor, but in FIG. 1, the APs are represented outside the information processing unit 48. The information processing unit 48 corresponds to a sub-interface module as viewed from the AP11, the AP12, the AP13, the AP14, and the AP15.

A buffer memory 50 is connected to the information processing unit 48. However, in FIG. 1, the buffer memory 50 is depicted in the information processing unit 48. The buffer memory 50 is a memory that temporarily stores the image data or the processing result data.

A communication unit 52 is connected to the information processing unit 48. The information processing unit 48 may function as the communication unit 52. The communication unit 42 and the communication unit 52 are connected to each other via the cable 43. As described above, the ultrasound diagnostic apparatus 10 and the external information processing apparatus 12 may be connected to each other via a network such as a wired LAN or a wireless LAN. A general-purpose communication protocol can be employed for the communication between the ultrasound diagnostic apparatus 10 and the external information processing apparatus 12. Streaming technology can be employed upon the transfer of a moving image data sequence. Data conforming to digital imaging and communications in medicine (DICOM) may be transferred. Electrocardiogramformation, three-dimensional position information, and the like may be sent from the ultrasound diagnostic apparatus 10 to the external information processing apparatus 12. In the shown configuration example, the external information processing apparatus 12 is not provided with a display device or an input device. Obviously, the external information processing apparatus 12 may be provided with those devices.

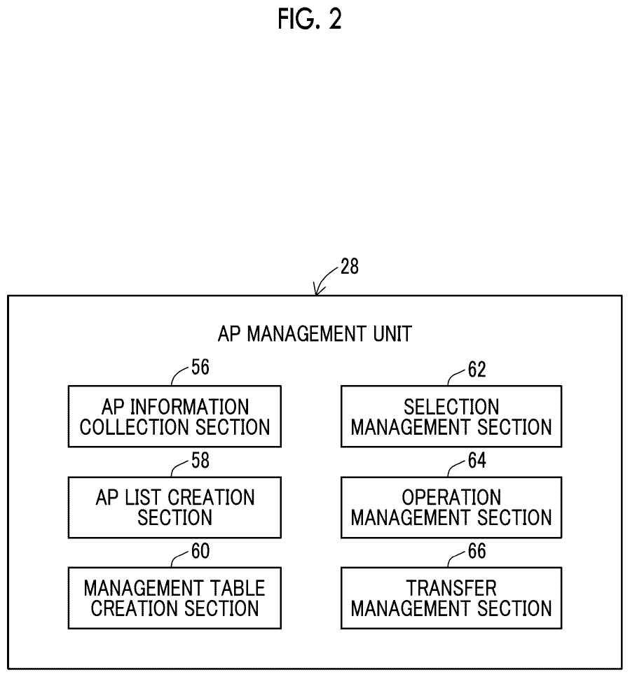

FIG. 2 shows a plurality of representative functions exhibited by the AP management unit 28 as a plurality of blocks 56 to 66. An AP information collection section 56 collects information (for example, an analysis type, an input specification, an output specification, and the like) required to manage each AP. An AP list creation section 58 creates the AP list based on the collected information. A management table creation section 60 creates a management table having the management information. The management information is information representing the interrelationships between the plurality of APs constituting the AP group and is information particularly representing a possibility of combination, a combination aspect, and the like.

A selection management section 62 manages the selection of the AP set based on the AP list and the management table. An operation management section 64 manages the operation of the AP set based on the AP list and the management table. Obviously, a single AP may be selected, and a single AP may be activated. A transfer management section 66 manages the data transfer from the ultrasound diagnostic apparatus 10 to the external information processing apparatus 12 and the data transfer from the external information processing apparatus 12 to the ultrasound diagnostic apparatus 10. The AP management unit 28 also has a function of collecting parameter information and status information necessary for operating the AP set.

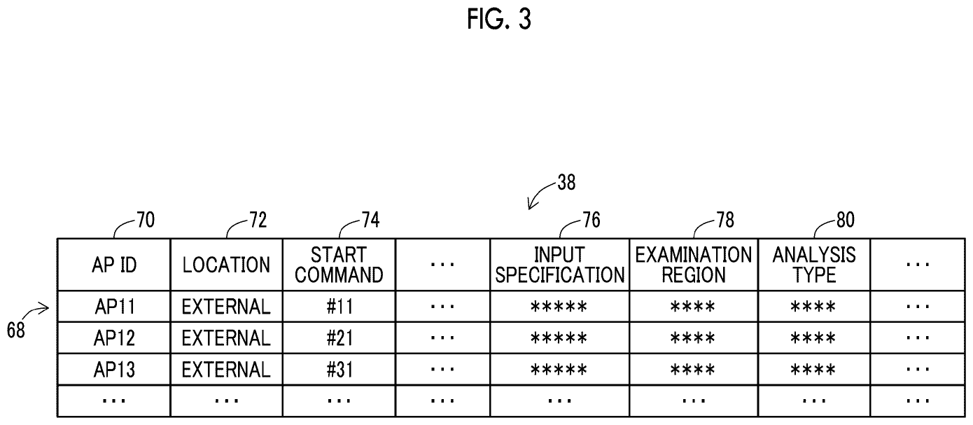

FIG. 3 shows an example of the AP list. The AP list 38 is composed of a plurality of rows 68 corresponding to the plurality of APs. Each row 68 includes an AP identifier 70, location information 72, a start command 74, and the like. The location information 72 is information for specifying a location where the AP is present and is specifically information representing the external information processing apparatus or information representing the ultrasound diagnostic apparatus. The external AP and the internal AP can be identified by referring to the location information 72. The start command 74 is a start command used to issue an instruction to activate the AP. An end command is also managed for each AP, but is not shown in FIG. 3.

Each row 68 also includes information 76 representing an input specification, information 78 representing an examination region, and information 80 representing an analysis type. The format or the type of data to be input to the AP can be specified by referring to the information 76. The examination region where the AP can be used can be specified by referring to the information 78. An examination site may be registered instead of the examination region. The function of the AP can be specified by referring to the information 80. Various kinds of information other than the information shown in FIG. 3 can be registered in the AP list 38. For example, for each AP, information regarding a probe that is compatible with the AP may be managed.

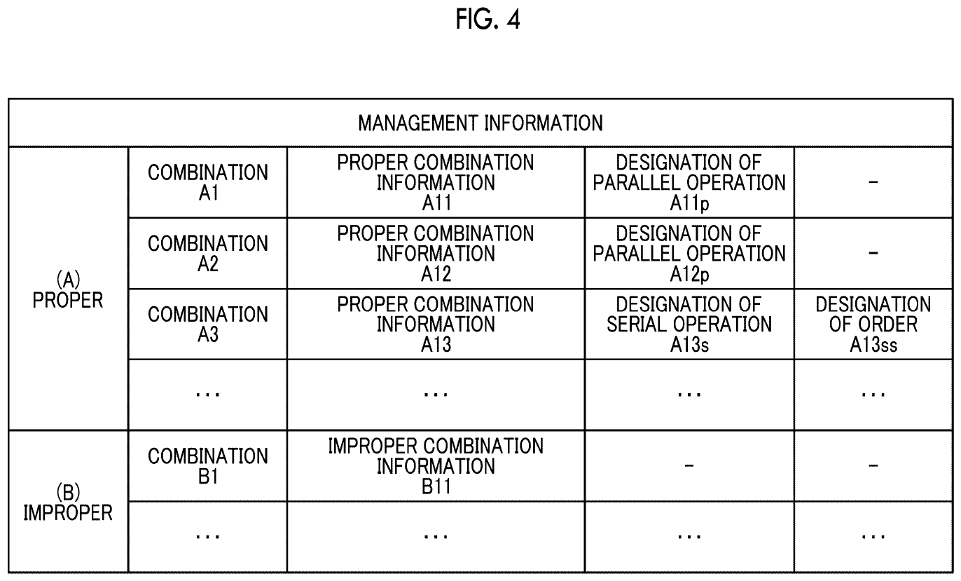

FIG. 4 shows a configuration example of the management information registered on the management table. The management information is information representing the interrelationships between the plurality of APs constituting the AP group. Specifically, a plurality of combinations (AP combinations) are defined based on the AP group. The plurality of combinations include a plurality of proper combinations A1, A2, and A3, and a plurality of improper combinations B1, . . . .

The proper combination is a combination that can be established, and the improper combination is a combination that cannot be established. For example, an AP to be used only in the ultrasound examination of the heart and an AP to be used only in the ultrasound examination of the abdomen correspond to the improper combination.

The management information includes pieces of proper combination information A11, A12, and A13 corresponding to the plurality of proper combinations A1, A2, and A3. The pieces of proper combination information A11, A12, and A13 each have information for specifying a plurality of APs constituting the proper combination. Improper combination information B11 has information for specifying a plurality of APs constituting the improper combination.

In the embodiment, the pieces of proper combination information A11, A12, and A13 each include designations A11p and A12p of the parallel operation or a designation A13s of the serial operation. The proper combination information A13 including the designation A13s of the serial operation further includes a designation A13ss of the connection order of the plurality of APs.

By preparing the above-described management information, through the reference to the management information, it is possible to quickly determine whether or not each individual combination is proper, and it is possible to quickly determine the attribute of the proper combination. Further, in a case in which the serial operation is designated as the attribute, the connection order of the APs can be quickly specified. The management information is composed of information input by the user. The management information may be automatically generated based on the AP list.

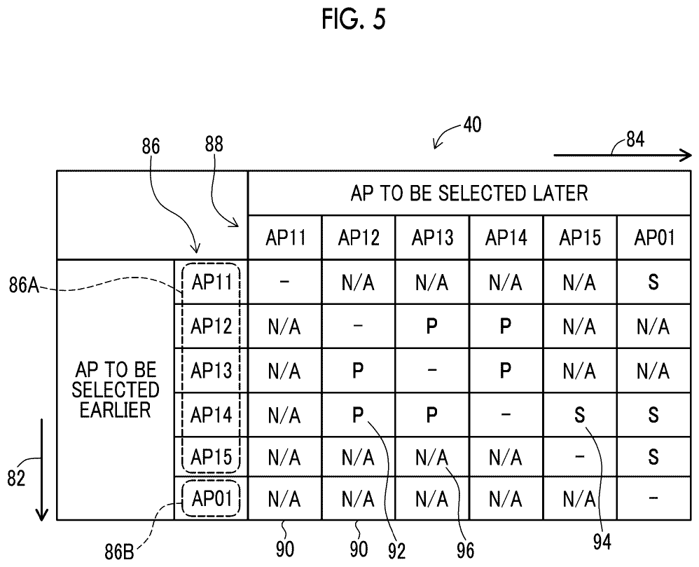

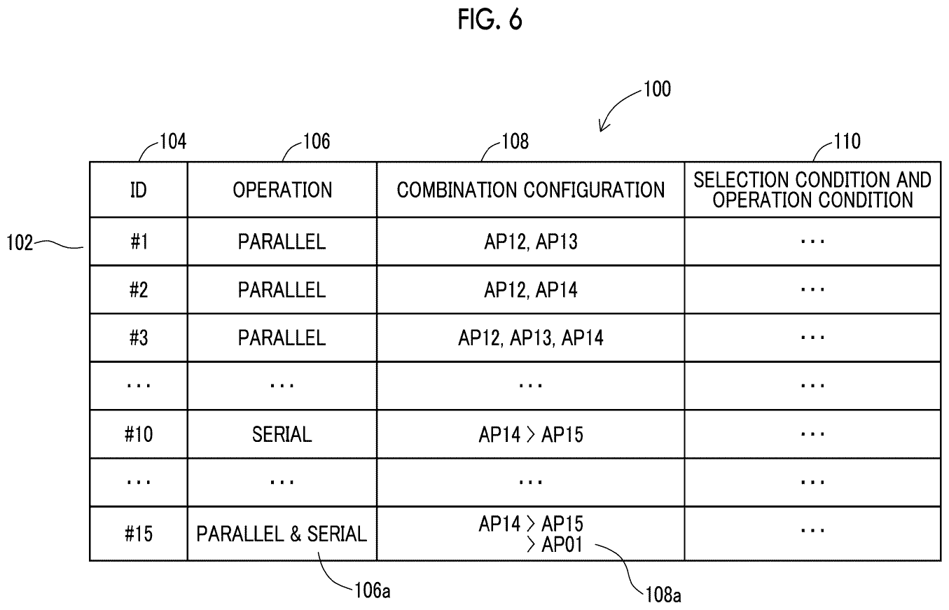

A specific example of the management table will be described with reference to FIGS. 5 and 6. FIG. 5 shows a first example of the management table. The management table 40 has a matrix structure having a vertical axis 82 and a horizontal axis 84. The matrix structure consists of a plurality of cells 90 arranged along the vertical axis 82 and the horizontal axis 84. More specifically, a plurality of AP candidates 86 to be selected earlier are assigned to the vertical axis 82, and a plurality of AP candidates 88 to be selected later are assigned to the horizontal axis 84. Among the plurality of AP candidates 86 and 88, the AP11 to the AP15 are each the external AP (refer to reference numeral 86A), and the AP01 is the internal AP (refer to reference numeral 86B).

The matrix structure includes the plurality of cells 90 corresponding to the plurality of AP pairs. The plurality of AP pairs are a plurality of AP combinations defined based on the AP group. In defining the plurality of APs, a selection order is also taken into consideration. Cell information is associated with each cell 90. The cell information includes information for specifying the AP to be selected earlier and information for specifying the AP to be selected later. In addition, the cell information includes information indicating whether or not the AP pair is a proper pair. A cell 90 corresponding to a proper pair may be referred to as a valid cell, and a cell 90 corresponding to an improper pair may be referred to as an invalid cell.

“P” (refer to reference numeral 92) registered in the cell 90 indicates that two APs corresponding to the cell are the proper AP pair and simultaneously, means the designation of the parallel operation. “S” (refer to reference numeral 94) registered in the cell 90 indicates that it is the proper AP pair and simultaneously, means the designation of the serial operation. In the serial operation, the AP selected earlier is an AP that operates earlier, and the AP selected later is the AP that operates later. “N/A” registered in the cell 90 indicates that it is the improper AP pair.

For example, assuming the management table 40 shown in FIG. 5 as a premise, in a case in which the AP12 is first selected and then the AP13 is selected, the AP14 can be further selected. In that case, the AP12, the AP13, and the AP14 operate in parallel. Meanwhile, in a case in which the AP14 is first selected and then the AP15 is selected, the AP01 can be further selected. In that case, the processing result of the AP14 is given to the AP15, and the processing result of the AP15 is given to the AP01. That is, the AP14-AP15-AP01 operate in series in the order of the arrangement thereof.

According to the management table 40 shown in FIG. 5, the selectable range can be easily narrowed down in accordance with the stepwise selection of the plurality of APs. In addition, since complex AP combinations are managed as sequences of the plurality of AP pairs, the scale of the management table 40 can be reduced.

FIG. 6 shows a second example of the management table. A management table 100 has a plurality of records 102 corresponding to the plurality of AP combinations. Each record 102 has an identifier 104 of the AP set, a parallel operation designation or serial operation designation 106, information 108 representing the plurality of APs constituting the AP set, information 110 representing a selection condition and an operation condition, and the like. By referring to the information 110, a special condition to be satisfied in a case of selecting a combination corresponding to the information 110 is specified, and a special operation condition in a case in which the combination corresponding to the information 110 is selected is specified.

Reference numeral 106a represents a composite designation. The composite designation includes the designation of the serial operation and the designation of the parallel operation. Specifically, as indicated by reference numeral 108a, the AP14 and the AP15 operate in series in the order of the arrangement thereof, and the AP14 and the AP01 operate in series in the order of the arrangement thereof. In a case of focusing on the AP15 and the AP01, the AP15 and the AP01 operate in parallel.

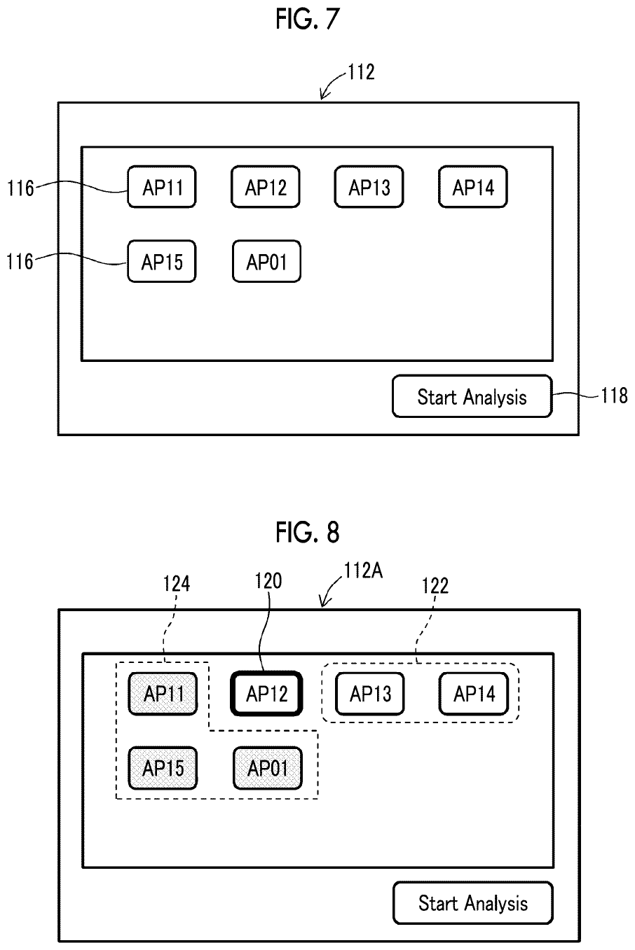

Next, the selection of the AP set will be described assuming the content of the management table shown in FIG. 5 as a premise. FIG. 7 shows an initial state 112 of the selection image according to the embodiment.

The selection image includes a plurality of display elements 116 representing the selectable AP11 to AP01. The selection image is displayed on, for example, a touch screen panel. Each display element 116 is a virtual button. A display element 118 is a start button that is operated after one or a plurality of APs are selected.

FIG. 8 shows a first state of the selection image. For example, in the initial state 112 shown in FIG. 7, in a case in which the AP12 is selected, a first state 112A shown in FIG. 8 occurs in accordance with the content of the management table shown in FIG. 5. In the first state 112A, a display element 120 is highlighted, and two display elements (refer to reference numeral 122) corresponding to the selectable AP13 and AP14 are displayed with high brightness. On the other hand, three display elements (refer to reference numeral 124) corresponding to the unselectable AP11, AP15, and AP01 are displayed with low brightness.

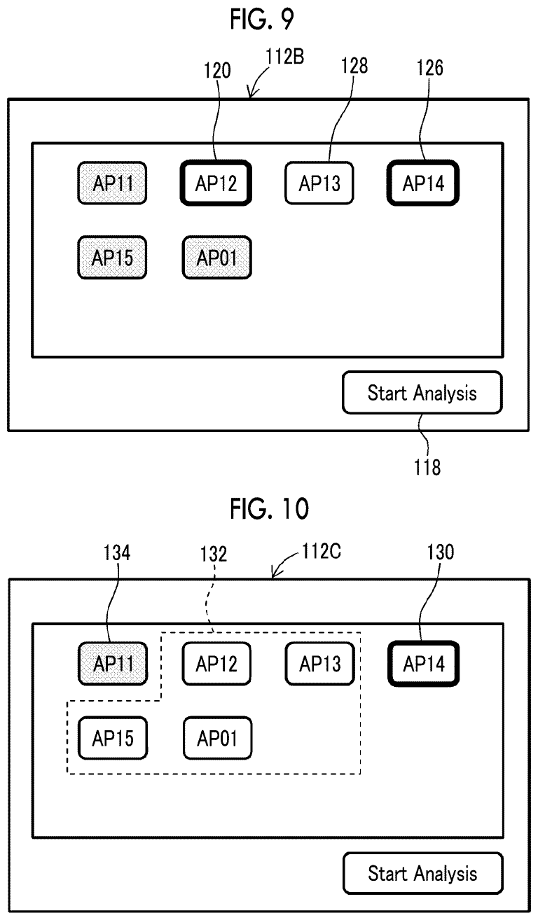

FIG. 9 shows a second state 112B of the selection image. In the first state 112A shown in FIG. 8, in a case in which a display element 126 corresponding to the AP14 is further selected, a display element 128 corresponding to the AP13 is displayed with high brightness as shown in FIG. 9 in accordance with the content of the management table shown in FIG. 5 (in fact, the display with high brightness is maintained). Consequently, the user can recognize that the AP13 can be selected subsequently. In a case in which the display element 128 corresponding to the AP13 is selected (the display element 128 is highlighted) and then the display element 118 is operated, the AP12, the AP13, and the AP14 operate in parallel in accordance with the content of the management table shown in FIG. 5.

Meanwhile, in the initial state 112 shown in FIG. 7, in a case in which a display element corresponding to the AP14 is selected, a third state 112C shown in FIG. 10 occurs in accordance with the content of the management table shown in FIG. 5. In the third state 112C, a display element 130 corresponding to the AP14 is highlighted, four display elements corresponding to the AP12, the AP13, the AP15, and the AP01 are displayed with high brightness (refer to reference numeral 132), and a display element 134 corresponding to the AP11 is displayed with low brightness.

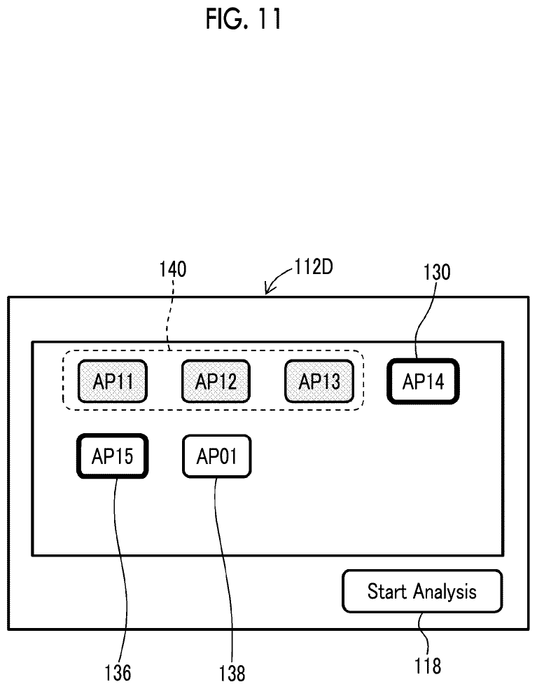

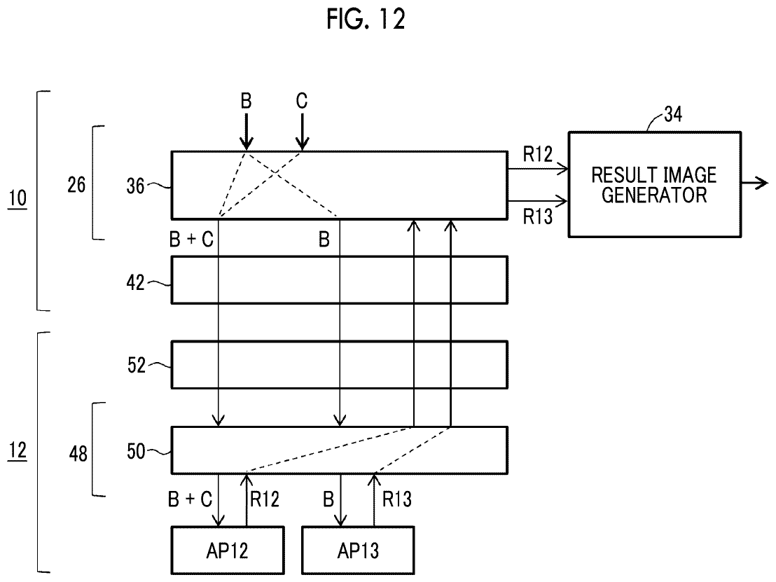

In the third state 112C, in a case in which a display element corresponding to the AP15 is further selected, as shown in FIG. 11, only the display of a display element 138 corresponding to the AP01 is maintained with high brightness, and the other three display elements are displayed with low brightness (refer to reference numeral 140), in accordance with the content of the management table shown in FIG. 5. In a case in which the display element 118 is operated at that point in time, the AP14 and the AP15 operate in series in the selection order thereof.

As described above, in the embodiment, the content of the selection image changes in a stepwise manner in accordance with the stepwise selection of the APs. Therefore, the AP selection of the user can be supported. In addition, it is possible to ensure that the proper combination is selected.

Next, an operation example of the AP set will be described by using FIGS. 12 to 14 assuming the content of the management table shown in FIG. 5 as a premise.

FIG. 12 shows the parallel operation of the AP12 and the AP13. As already described, the ultrasound diagnostic apparatus 10 includes the information processing unit 26, and the information processing unit 26 includes the AP management unit. Reference numeral 36 indicates the memory managed by the AP management unit. The information processing unit 26 includes the result image generator 34. Meanwhile, the external information processing apparatus 12 includes the information processing unit 48. The buffer memory 50 is managed by the information processing unit 48. The communication unit 42 in the ultrasound diagnostic apparatus 10 and the communication unit 52 in the external information processing apparatus 12 communicate with each other.

The AP12 is an AP for executing analysis based on the tomographic image data sequence and the blood flow image data sequence. For example, the AP12 generates, for each image data pair (tomographic image data and blood flow image data), a two-dimensional vector image based on the image data pair. The two-dimensional vector image is composed of a plurality of two-dimensional vectors indicating the motion of the blood flow in the heart. The AP13 is an AP for executing analysis based on the tomographic image data sequence. For example, the AP13 tracks a plurality of points set in the myocardium based on the tomographic image data sequence to generate a moving image showing the motion of the myocardium. The AP12 and the AP13 operate independently of each other without cooperating with each other, that is, without exchanging data, unlike in the case of the serial operation.

As shown in FIG. 12, a tomographic image data sequence B is temporarily stored in the memory 36. A blood flow image data sequence C is also temporarily stored in the memory 36. The tomographic image data sequence B and the blood flow image data sequence C read from the memory 36 are sent to the buffer memory 50 via the communication unit 42 and the communication unit 52 and are temporarily stored in the buffer memory 50. The tomographic image data sequence B and the blood flow image data sequence C read from the buffer memory 50 are supplied to the AP12. Each individual AP may access the buffer memory 50 to acquire data necessary for each AP.

Meanwhile, the tomographic image data sequence B read separately from the memory 36 is sent to the buffer memory 50 via the communication unit 42 and the communication unit 52 and is temporarily stored in the buffer memory 50. The tomographic image data sequence B read from the buffer memory 50 is supplied to the AP13. Instead of two transfers of the tomographic image data sequence B, the tomographic image data sequence B read from the buffer memory 50 may be supplied in parallel to the AP12 and the AP13.

The AP12 executes the analysis based on the tomographic image data sequence B and the blood flow image data sequence C and generates an analysis result data sequence R12. The analysis result data sequence R12 consists of a plurality of pieces of analysis result data arranged in time series order, and each individual piece of the analysis result data is image data.

The analysis result data sequence R12 is temporarily stored in the buffer memory 50. The analysis result data sequence R12 read from the buffer memory 50 is sent to the memory 36 via the communication units 52 and 42 and is stored in the memory 36. The analysis result data sequence R12 read from the memory 36 is sent to the result image generator 34.

The AP13 executes the analysis based on the tomographic image data sequence B and generates an analysis result data sequence R13. The analysis result data sequence R13 consists of a plurality of pieces of analysis result data arranged in time series order, and each individual piece of the analysis result data is image data.

The analysis result data sequence R13 is temporarily stored in the buffer memory 50. The analysis result data sequence R13 read from the buffer memory 50 is sent to the memory 36 via the communication units 52 and 42 and is stored in the memory 36. The analysis result data sequence R13 read from the memory 36 is sent to the result image generator 34.

The result image generator 34 generates a result image data sequence based on the analysis result data sequence R12 and the analysis result data sequence R13. The result image data sequence is sent to the main display. The result image data sequence is composed of a plurality of pieces of result image data arranged in time series order. Each individual piece of the result image data includes first image data representing the analysis result of the AP12 and second image data representing the analysis result of the AP13.

The main display displays the result image sequence as a moving image. In a case in which the juxtaposed display mode is selected as the display mode, each individual result image includes a first image and a second image that are disposed in a juxtaposed manner. In a case in which the composite display mode is selected as the display mode, each individual result image includes a composite image generated by combining the first image and the second image.

In a case in which the image data sequence is transferred from the ultrasound diagnostic apparatus 10 to the external information processing apparatus 12, a parameter group required for the analysis of the image data sequence is also transferred. The parameter group includes, for example, a frame rate, a diagnostic depth range, a gain, a focus depth, and the like. The image data sequence before the coordinate transformation may be transferred from the ultrasound diagnostic apparatus 10 to the external information processing apparatus. In addition, the image data corresponding to the still image may be transferred instead of the image data sequence corresponding to the moving image. For example, a plurality of pieces of image data sequentially selected from the stored image data sequence may be sequentially transferred. In that case, the analysis by the external AP is carried out for each piece of the transferred image data.

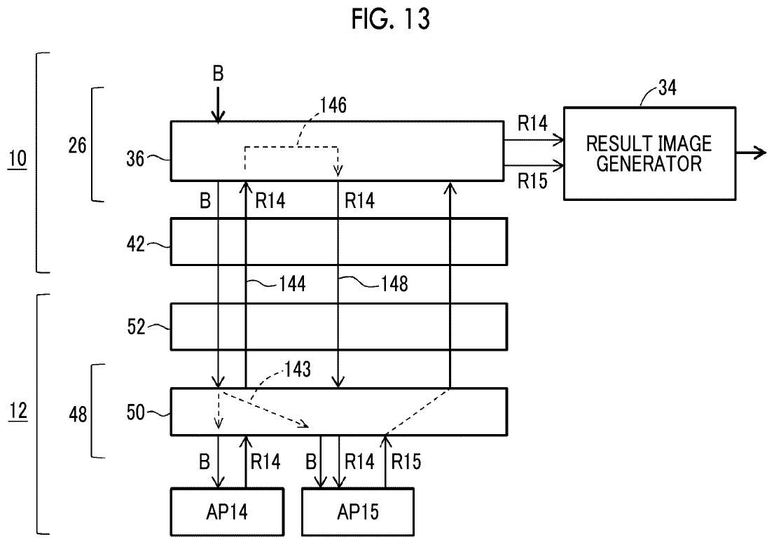

FIG. 13 shows a first example of the serial operation of the AP14 and the AP15. In FIG. 13, the same elements as the elements shown in FIG. 12 are designated by the same reference numerals, and the description thereof will be omitted.

The AP14 and the AP15 operate in series in that order. The AP14 performs the analysis based on the tomographic image data sequence B. The AP15 performs the analysis based on an analysis result data sequence R14 of the AP14. In this manner, the AP14 and the AP15 have a data exchange relationship, and the AP14 and the AP15 operate in cooperation with each other.

For example, the AP14 detects a lesion part included in each individual tomographic image, and the AP15 evaluates the detected lesion part. In a case in which such a configuration is employed, lesion part information (position information and size information) that is the analysis result of the AP14 is sent to the AP15 for each tomographic image. The AP15 evaluates a partial image specified by the lesion part information and calculates a plurality of scores corresponding to a plurality of types of evaluation items. The AP15 outputs analysis result data consisting of numerical information, for each tomographic image.

Specifically, the tomographic image data sequence B is sent to the AP14 via the memory 36, the communication unit 42, the communication unit 52, and the buffer memory 50. The tomographic image data sequence B is also sent to the AP15 as necessary. In that case, the tomographic image data sequence B temporarily stored in the buffer memory 50 is sent in parallel to the AP14 and the AP15 (refer to reference numeral 143).

The analysis result data sequence R14 is output from the AP14 through the analysis of the tomographic image data sequence B by the AP14. In the first example shown in FIG. 13, the analysis result data sequence R14 is temporarily stored in the memory 36 via the buffer memory 50, the communication unit 52, and the communication unit 42 (refer to reference numeral 144). The analysis result data sequence R14 read from the memory 36 is sent to the AP15 via the communication unit 42, the communication unit 52, and the buffer memory 50 (refer to reference numerals 146 and 148). The analysis result data sequence R14 read separately from the memory 36 is sent to the result image generator 34.

In the AP15, the analysis based on the analysis result data sequence R14 is carried out. In that case, the tomographic image data sequence B is referred to as necessary. An analysis result data sequence R15 output from the AP15 is temporarily stored in the memory 36 via the buffer memory 50, the communication unit 52, and the communication unit 42. The analysis result data sequence R15 read from the memory 36 is sent to the result image generator 34.

The result image generator 34 generates the result image data sequence based on the analysis result data sequence R14 and the analysis result data sequence R15 and outputs the result image data sequence to the main display. The result image data sequence is generated in accordance with a display mode selected by the user.

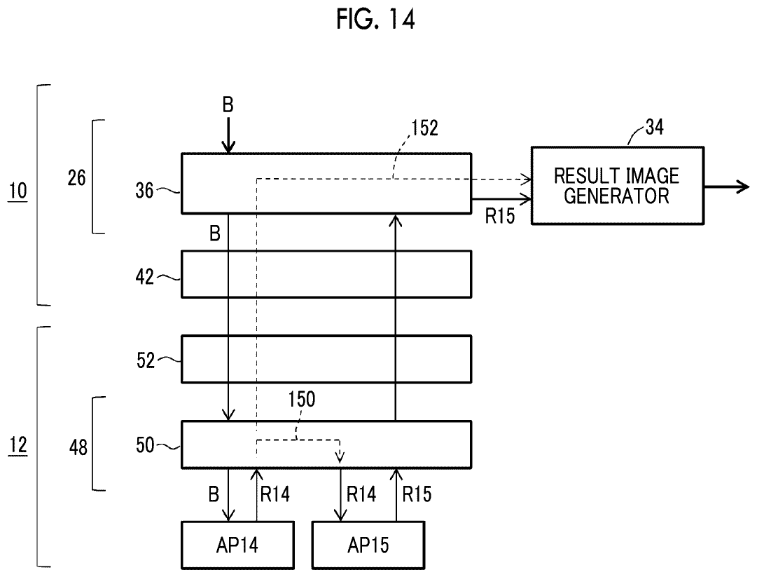

FIG. 14 shows a second example of the serial operation of the AP14 and the AP15. In FIG. 14, the same elements as the elements shown in FIG. 12 are designated by the same reference numerals, and the description thereof will be omitted.

The tomographic image data sequence B is sent to the AP14 via the memory 36, the communication unit 42, the communication unit 52, and the buffer memory 50. The tomographic image data sequence B is also sent to the AP15 as necessary.

The analysis result data sequence R14 is output from the AP14 through the analysis of the tomographic image data sequence B by the AP14. In the second example shown in FIG. 14, the analysis result data sequence R14 is directly sent to the AP15 via the buffer memory 50 (refer to reference numeral 150). The analysis result data sequence R14 is temporarily stored in the memory 36 via the buffer memory 50, the communication unit 52, and the communication unit 42 as necessary. In that case, the analysis result data sequence R14 read from the memory 36 is sent to the result image generator 34 (refer to reference numeral 152).

In the AP15, the analysis based on the analysis result data sequence R14 is carried out. In that case, the tomographic image data sequence B is referred to as necessary. The analysis result data sequence R15 output from the AP15 is temporarily stored in the memory 36 via the buffer memory 50, the communication unit 52, and the communication unit 42. The analysis result data sequence R15 read from the memory 36 is sent to the result image generator 34.

In the second example shown in FIG. 14, the transfer of the analysis result data sequence R14 from the ultrasound diagnostic apparatus 10 to the external information processing apparatus 12 is not necessary. Therefore, an advantage of reducing the amount of data to be transferred can be obtained.

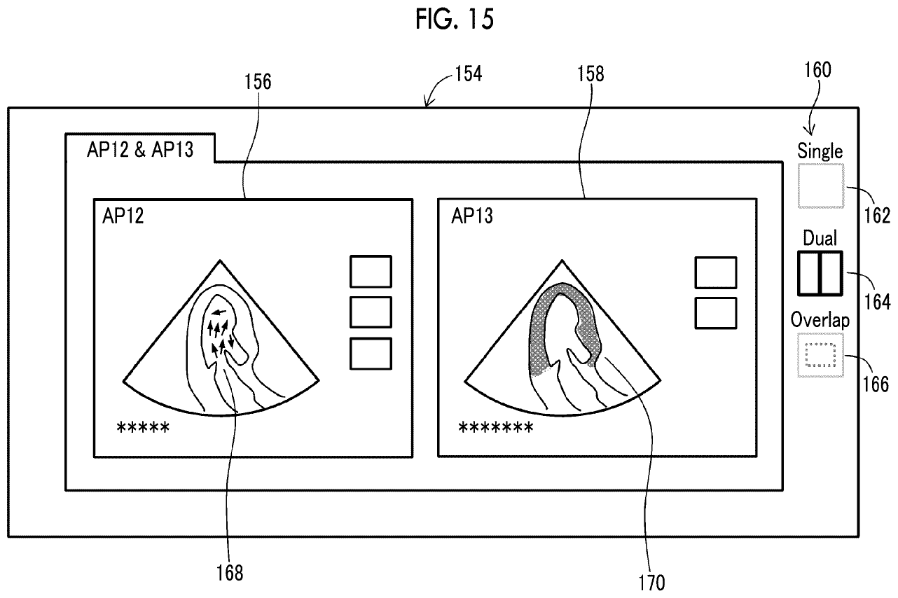

FIG. 15 shows a first example of the result image generated by the result image generator. In accordance with the selection of the juxtaposed display mode, a result image 154 includes an image 156 showing a first analysis result and an image 158 showing a second analysis result. The image 156 and the image 158 are arranged in a horizontal direction. More specifically, the image 156 shows the analysis result of the AP12 and includes a plurality of vectors 168. The image 158 shows the analysis result of the AP13 and includes a band-shaped colored portion 170 indicating myocardial motion.

A display mode selection field 160 includes a button 162 for selecting a single display mode in which the analysis result is individually displayed, a button 164 for selecting the juxtaposed display mode in which a plurality of analysis results are displayed in a juxtaposed manner, and a button 166 for selecting a composite display mode in which the plurality of analysis results are combined (overlapped) and displayed. In FIG. 15, the button 164 is selected.

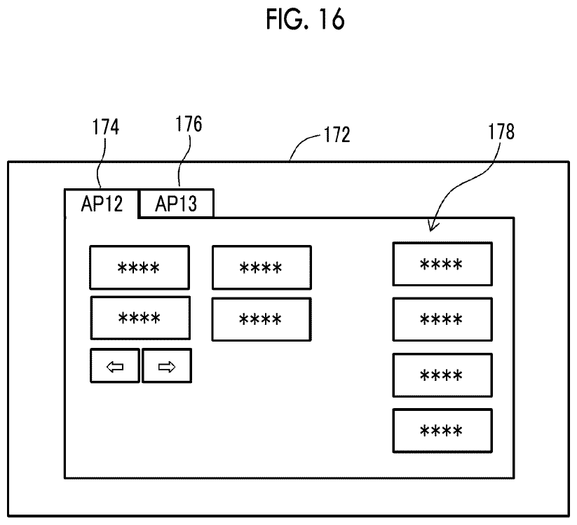

FIG. 16 shows a first example of the operation image generated by the operation image generator. As a content of an operation image 172, either a first sheet 174 or a second sheet 176 is selected. In the shown example, the first sheet 174 includes a plurality of buttons 178 for issuing instructions to the AP12. The second sheet 176 also includes a plurality of buttons for issuing instructions to the AP13.

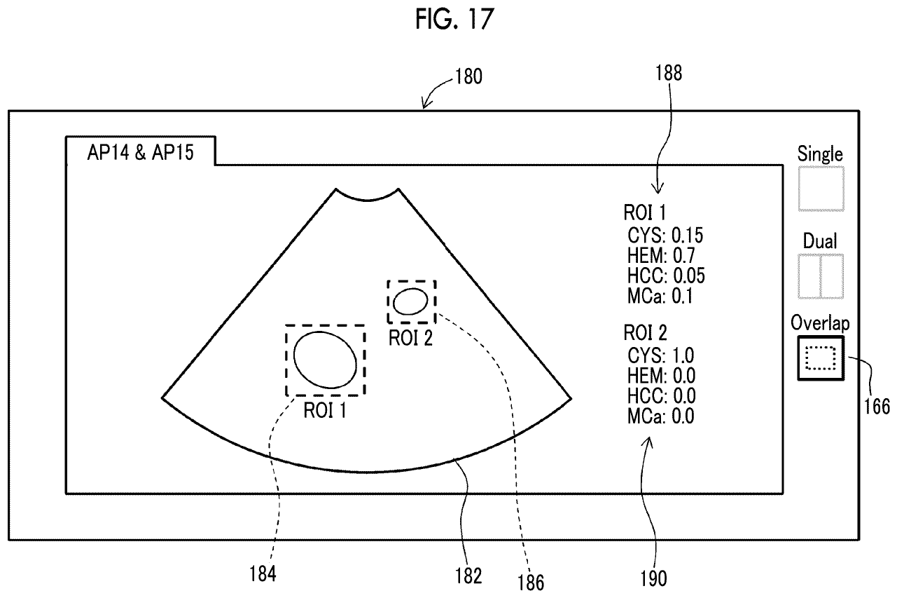

FIG. 17 shows a second example of the result image generated by the result image generator. In accordance with the selection of the composite display mode, the composite image representing the first analysis result and the second analysis result is generated, and the composite image is displayed as a result image 180. The button 166 corresponding to the composite display mode is selected.

The result image 180 includes a tomographic image 182 and two figures 184 and 186 indicating two ROIs. The two figures 184 and 186 surround two lesion parts specified by the image analysis. The figures 184 and 186 or information for generating the figures 184 and 186 correspond to the analysis result of the AP14. The analysis result of the AP14 may include the tomographic image 182.

The result image 180 includes first numerical information 188 and second numerical information 190. The first numerical information 188 indicates a result of the analysis performed on a first lesion part surrounded by the figure 184 and specifically, consists of a plurality of scores corresponding to a plurality of types of evaluation items. The second numerical information 190 indicates a result of the analysis performed on a second lesion part surrounded by the figure 186 and specifically, consists of a plurality of scores corresponding to a plurality of types of evaluation items.

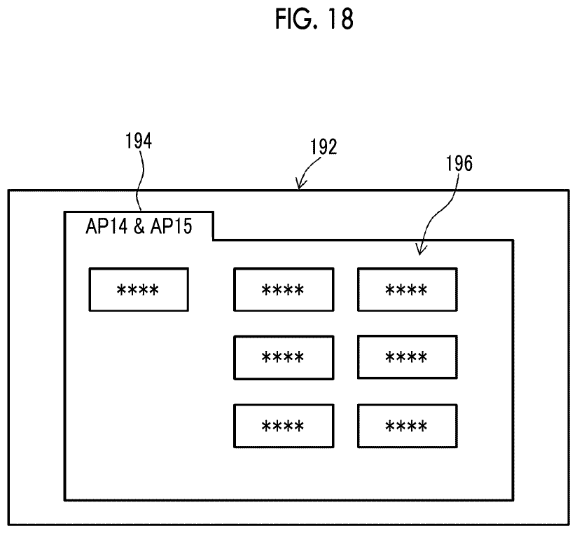

FIG. 18 shows a second example of the operation image generated by the operation image generator. An operation image 192 includes a single sheet 194, and the sheet 194 includes a plurality of buttons 196. The plurality of buttons 196 are used to issue instructions to the AP14 and the AP15 in the shown example. The plurality of buttons 196 may include a common button for issuing an instruction to both the AP14 and the AP15.

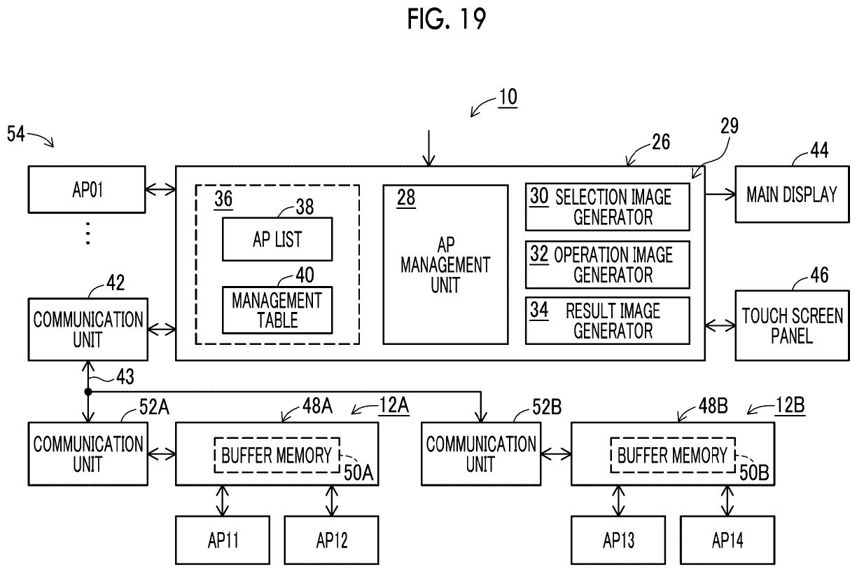

FIG. 19 shows an ultrasound diagnostic system according to another embodiment. In FIG. 19, the same elements as the elements shown in FIG. 1 are designated by the same reference numerals, and the description thereof will be omitted. In FIG. 19, the ultrasound probe, the transmission circuit, the reception circuit, the data processing circuit, and the image generation unit are not shown.

The ultrasound diagnostic system is composed of the ultrasound diagnostic apparatus 10, a first external information processing apparatus 12A, and a second external information processing apparatus 12B. The ultrasound diagnostic apparatus 10, the first external information processing apparatus 12A, and the second external information processing apparatus 12B are connected to each other via the cable 43. The first external information processing apparatus 12A includes an information processing unit 48A, a communication unit 52A, the AP11, the AP12, and a buffer memory 50A. The second external information processing apparatus 12B includes an information processing unit 48B, a communication unit 52B, the AP13, the AP14, and a buffer memory 50B. An AP group 54A is composed of the plurality of external APs (the AP11 to the AP14) and one internal AP (AP01).

Even in a case in which the configuration shown in FIG. 19 is employed, the AP management unit 28 manages the collective selection of the plurality of APs to function together during the ultrasound examination and manages the simultaneous operation or the cooperative operation of the plurality of APs, based on the management information shown in FIGS. 4 to 6. The ultrasound diagnostic apparatus 10, the first external information processing apparatus 12A, and the second external information processing apparatus 12B may be connected to a server via a network such as a LAN.

In the above description, the AP set is composed of the plurality of external APs, but the AP set may be composed of the external AP and the internal AP. In any case, the AP management unit 28 manages the selection and the operation of the AP set based on the management information shown in FIGS. 4 to 6.

What is claimed is:

1. An ultrasound diagnostic system comprising:
an ultrasound diagnostic apparatus that generates image data based on reception data;
an external information processing apparatus connected to the ultrasound diagnostic apparatus;
an application program (AP) group provided in the external information processing apparatus or provided across the ultrasound diagnostic apparatus and the external information processing apparatus, and composed of a plurality of APs for processing the image data; and
a management unit that manages selection of an AP set from the AP group and manages an operation of the AP set, based on management information representing interrelationships between the plurality of APs constituting the AP group,
wherein the AP set consists of a plurality of APs that operate together during an ultrasound examination.

2. The ultrasound diagnostic system according to claim 1,
wherein the management information includes one or a plurality of pieces of proper combination information, and
each piece of the proper combination information includes information for specifying the plurality of APs that operate together.

3. The ultrasound diagnostic system according to claim 2,
wherein each piece of the proper combination information further includes a designation of a parallel operation or a designation of a serial operation, and
the management unit
causes the plurality of APs constituting the AP set to perform the parallel operation in accordance with the designation of the parallel operation, and
causes the plurality of APs constituting the AP set to perform the serial operation in accordance with the designation of the serial operation.

4. The ultrasound diagnostic system according to claim 3,
wherein the proper combination information including the designation of the serial operation includes information indicating a connection order of the plurality of APs constituting the AP set, and
the management unit connects the plurality of APs constituting the AP set in series in accordance with the information indicating the connection order.

5. The ultrasound diagnostic system according to claim 2,
wherein the management information has a matrix structure,
the matrix structure includes a plurality of cells corresponding to a plurality of AP pairs, which are a plurality of combinations defined based on the AP group, and
the plurality of cells consist of one or a plurality of valid cells associated with the one or the plurality of pieces of proper combination information and one or a plurality of invalid cells other than the one or the plurality of valid cells.

6. The ultrasound diagnostic system according to claim 5,
wherein the matrix structure has a first axis to which a plurality of AP candidates to be selected earlier are assigned and a second axis to which a plurality of AP candidates to be selected later are assigned, and
the management unit identifies a specific cell based on an AP selected earlier and an AP selected later in the matrix structure and refers to information associated with the specific cell.

7. The ultrasound diagnostic system according to claim 1,
wherein the management unit connects, in controlling a serial operation of the plurality of APs constituting the AP set, the plurality of APs constituting the AP set in series in an order according to the management information so that a processing result output from an i-th AP is input to an i+1-th AP.

8. The ultrasound diagnostic system according to claim 7,
wherein the ultrasound diagnostic apparatus includes the management unit, and
the management unit receives processing result data output from the i-th AP and transfers the processing result data to the i+1-th AP.

9. The ultrasound diagnostic system according to claim 7,
wherein the external information processing apparatus includes a buffer memory, and
the management unit manages an operation of the external information processing apparatus such that, in a case in which the i-th AP and the i+1-th AP belong to the external information processing apparatus, the processing result data output from the i-th AP is written into the buffer memory and the processing result data read from the buffer memory is input to the i+1-th AP.

10. The ultrasound diagnostic system according to claim 1, further comprising:
a selection image generation unit that generates a selection image for selecting the AP set from the AP group, based on the management information.

11. The ultrasound diagnostic system according to claim 10,
wherein the selection image includes one or a plurality of display elements indicating one or a plurality of AP candidates that are currently selectable, and
a content of the selection image changes in a stepwise manner in a process of selecting a plurality of APs from the AP group in a stepwise manner.

12. The ultrasound diagnostic system according to claim 1, further comprising:
a display controller that controls display of a plurality of pieces of processing result data output from the plurality of APs constituting the AP set,
wherein the display controller
displays the plurality of pieces of processing result data in a juxtaposed manner in a case in which a juxtaposed display mode is selected, and
displays the plurality of pieces of processing result data in a composite manner in a case in which a composite display mode is selected.

13. An ultrasound diagnostic apparatus connected to an external information processing apparatus, the ultrasound diagnostic apparatus comprising:
a generation unit that generates image data based on reception data; and
a management unit that manages an application program (AP) group composed of a plurality of APs for processing the image data,
wherein the AP group is provided in the external information processing apparatus or is provided across the ultrasound diagnostic apparatus and the external information processing apparatus,
the management unit manages selection of an AP set from the AP group and manages an operation of the AP set, based on management information representing interrelationships between the plurality of APs constituting the AP group, and
the AP set consists of a plurality of APs that operate together during an ultrasound examination.

14. A non-transitory computer readable medium storing a program executed in an ultrasound diagnostic system, the ultrasound diagnostic system including an ultrasound diagnostic apparatus that generates image data based on reception data, and an external information processing apparatus connected to the ultrasound diagnostic apparatus, in which an application program (AP) group composed of a plurality of APs for processing the image data is provided in the external information processing apparatus or is provided across the ultrasound diagnostic apparatus and the external information processing apparatus, the program comprising:
a function of managing selection of an AP set from the AP group based on management information representing interrelationships between the plurality of APs constituting the AP group; and
a function of managing an operation of the AP set based on the management information,
wherein the AP set consists of a plurality of APs that operate together during an ultrasound examination.
